(19) United States
(12) Patent Application Publication
(10) Pub. No.: US 2011/0087263 A1
(43) Pub. Date: Apr. 14, 2011

(54) External Pneumatic Compression Device

(76) Inventor: Patrick Thomas Arber, Hockessin, DE (US)

(21) Appl. No.: 12/904,133

(22) Filed: Oct. 13, 2010

Related U.S. Application Data
(60) Provisional application No. 61/251,152, filed on Oct. 13, 2009.

(51) Int. Cl. A61B 17/00

(52) U.S. Cl. 606/202

(57) ABSTRACT

The present invention relates to a device for facilitating a colonoscopy, and more particularly to a non-invasive external pneumatic compression device that applies pressure to the abdomen of a patient in order to decrease looping of the colon during a colonoscopy and thus effectively splint a looping colon without the need for human intervention. This external pneumatic compression device comprises a flexible and breathable elastic abdominal binder for surrounding the abdomen of a patient undergoing a colonoscopy, a counter pressure plate that is secured to the middle inside of the binder for providing a counter pressure during and while the bladder or bladders are inflated, a means for supplying air and regulating the pressure in the bladders, and a plurality of inflatable bladders for applying downward pressure to specific anatomical areas of the large intestines of a patient when inflated. When the binder is secured to the abdomen of a patient, the inflatable bladders will be placed over four noted regions of the abdomen corresponding to the ascending colon, the mid-umbilical colon, the transverse colon, and the sigmoid bladder.

### REFERENCE TO RELATED APPLICATIONS

This patent application claims the benefit of U.S. Provisional Application No. 61/251,152 filed on Oct. 13, 2009, the disclosure of which is incorporated herein by reference in its entirety.

### BACKGROUND OF THE INVENTION

1. Field of the Invention

The present invention relates to a device for facilitating a colonoscopy, and more particularly to an external pneumatic compression device that splints a looping colon for easing the passage of a colonoscope during a colonoscopy.

2. Description of Related Art

Colonoscopy is a procedure used to examine the inside of the colon and rectum. Colonoscopy can detect inflamed tissue, ulcers, and abnormal growths. The procedure is used to look for early signs of colorectal cancer and can help doctors diagnose unexplained changes in bowel habits, abdominal pain, bleeding from the anus, and weight loss.

During a colonoscopy, a patient lies on his or her side or back while the doctor slowly advances a flexible tube with a camera at the end (colonoscope) through the patient's large intestine to examine the lining. Once the colonoscope has reached the opening to the small intestine, it is slowly withdrawn and the lining of the large intestine is carefully examined. However, looping of the colonoscope is a common challenge that results in discomfort and pain for the patient and hinders advancement of the colonoscope. Although medical technology has advanced at a lightning pace, a patient undergoing a colonoscopy still experiences a lot of discomfort during the examination. In order to overcome looping, it is common for a nurse or an assistant to compress the abdomen during insertion of colonoscope in an attempt to support the colon. It is desirable to have a device that can apply consistent pressure to patient's abdomen and support the colon during the insertion of colonoscope.

The use of a device and method to compress a patient's abdomen and facilitate a colonoscopy is known in the prior art. More specifically, by way of example, WO 96/14811 to Paternuosto, et. al. discloses an external straightener for a colonoscopy that has an ovoid shape with a protuberance in proximity to one of the two ends. This device utilizes the patient's own weight in the various positions of use; thus the pressure exerted on the patient's belly is correlated to the patient's weight. The present invention is distinguished from the prior art in that the present invention further comprises an external air supply device and a plurality of bladders that can selectively and simultaneously compress multiple regions of abdomen during a colonoscopy.

U.S. Pat. No. 5,685,321 to Klingenstein discloses a device and method to facilitate a colonoscopy by externally compressing the colon. The device is a corset-like wrap which embraces a patient's abdomen and has an inflatable bladder to apply force to the patient's abdomen. The inflatable bladder is placed on the wrap so that it covers the lower left-hand quadrant of the patient's abdomen. This placement allows the sigmoid colon to be supported.

U.S. Pat. No. 6,672,311 to Rindfleish discloses a vest including inflatable bladders of which the pressure can be adjusted to the desired amount to aid the doctor in performing a colonoscopy examination.

The present invention is distinguished from the aforementioned two prior art in that the present invention further comprises a counter pressure plate which is a hard, supportive plate. The counter pressure plate provides counter pressure during and while the inflatable bladders are inflated. Moreover, because the inflatable bladders are connected to the binder/plate through four ports which are located at 12, 9, 3 o'clock positions and the center of the plate, when the binder is secured to the abdomen of a patient, the inflatable bladders will be placed over four noted regions of the abdomen corresponding to the ascending colon, the mid-umbilical colon, the transverse colon, and the sigmoid bladder. The present invention can selectively and simultaneously compress multiple critical regions of abdomen to enhance the passage of a colonoscope during a colonoscopy.

### SUMMARY OF THE INVENTION

Broadly, an embodiment of the present invention generally relates to an external pneumatic compression device that splints a looping colon for easing the passage of a colonoscope during a colonoscopy.

Embodiments of the present invention include a non-invasive external pneumatic compression device that applies pressure to the abdomen of a patient in order to decrease looping of the colon during a colonoscopy and thus effectively splint a looping colon without the need for human intervention.

In one embodiment of the present invention, the external pneumatic compression device comprises a flexible and breathable elastic abdominal binder with a fastening means on each end, a counter pressure plate with four ports, a plurality of inflatable bladders, one air control/release valve attached to each of the respective inflatable bladders, and one manometer to supply air to each of the respective inflatable bladders.

The abdominal binder is used to surround the abdomen of a patient undergoing a colonoscopy. The binder can be approximately 45 to 96 inches in length and approximately 4 to 16 inches in width. In one preferred embodiment, the width of the binder is about 6 inches.

The function of the counter pressure plate is to provide counter pressure during and while one or more inflatable bladders are inflated. Thus, the counter pressure plate is a hard, supportive plate made out of metal, cardboard, or preferably plastic. It can measure between 2″×2″ to 8″×8″. In one preferred embodiment, the counter pressure plate is about 6″×6″.

In some embodiments, there are four ports placed at the 12, 9, 3 o'clock positions and the center of the plate. The locations of these ports ensure that the inflatable bladders will be placed over the critical regions of a patient's abdomen.

An inflatable bladder can be of any shape, such as round, square, rectangular, oval, oblong, or triangular, can hold air or liquid, and can be made of medical grade PVC, rubber, or plastic, but preferably non-latex rubber between 1″ to 8″.

In other embodiments, the compression device further comprises bladder sleeve pockets to accommodate the inflatable bladders.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a transparent view of an abdominal binder in accordance with an embodiment of the present invention.

FIG. 2 is a transparent view of an abdominal binder and an attached counter pressure plate where four ports are placed at the 12, 9, 3 o'clock positions and the center of the plate in accordance with an embodiment of the present invention.

FIG. 3 is a transparent view of an abdominal binder, a counter pressure plate and a plurality of bladder sleeve pockets in accordance with an embodiment of the present invention.

FIG. 4 is a transparent view of an abdominal binder, a counter pressure plate, and a plurality of inflatable bladders inserted into the plurality of bladder sleeve pockets in accordance with an embodiment of the present invention.

FIG. 5 is a transparent view of the abdominal binder, the counter pressure plate, and the inflated bladders inside the bladder sleeve pockets in accordance with an embodiment of the present invention in operation position. While the bladders are inflated, large intestine is maneuvered into shape to accept advancing colonoscope and to decrease and stabilize looping throughout procedure.

FIG. 6 is a transparent view of the external pneumatic compression device in accordance with an embodiment of the present invention. FIG. 6 illustrates the connection among the inflatable bladders, the counter pressure plate, the abdominal binder, and a manometer.

FIG. 7 is a side view of the external pneumatic compression device in accordance with an embodiment of the present invention from upper body of a patient. FIG. 7 demonstrates the inflatable bladders, the counter pressure plate, the abdominal binder, and a manometer from a side view.

FIG. 8 is a side view of the external pneumatic compression device in accordance with an embodiment of the present invention from upper body of a patient. FIG. 8 demonstrates the connection among the inflatable bladders, the counter pressure plate, the abdominal binder, and a manometer from a side view.

### DESCRIPTION OF THE PREFERRED EMBODIMENT

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

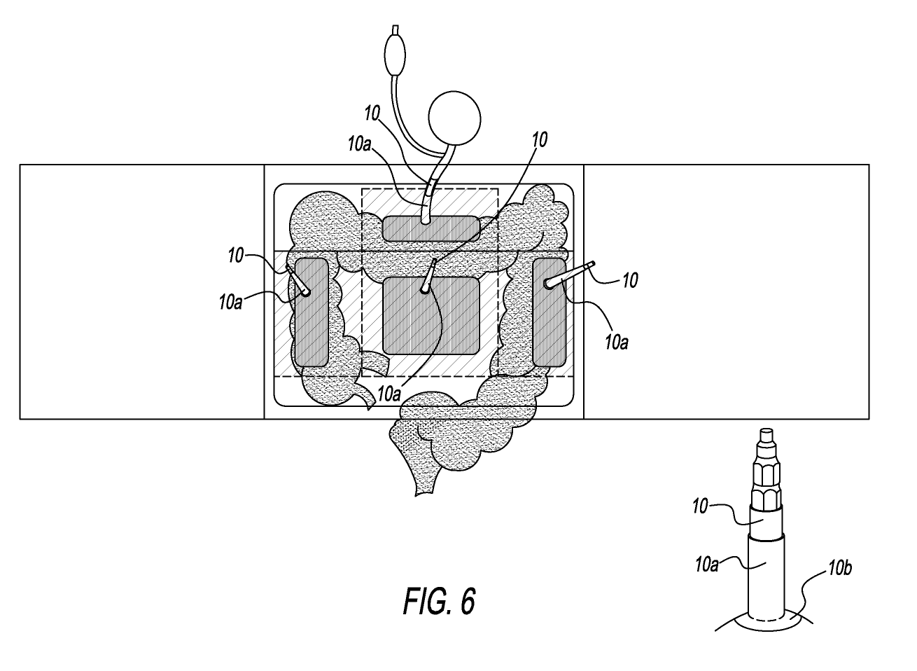

Referring to FIG. 6, there is disclosed an external pneumatic compression device that splints a looping colon for easing the passage of a colonoscope during a colonoscopy. An external pneumatic compression device in accordance with an embodiment of the present invention in operation position is shown in FIG. 6. The external pneumatic compression device 100 comprises a flexible and breathable elastic abdominal binder 1 with a fastening means 2 on each end, a counter pressure plate 3 with four ports 4 for each of the respective inflatable bladders 6, 7, 8, 9, one air control/release valve 10 attached to the tube 10a at port 10b on each of the respective inflatable bladders, and one manometer 11 with tubing and a bulb.

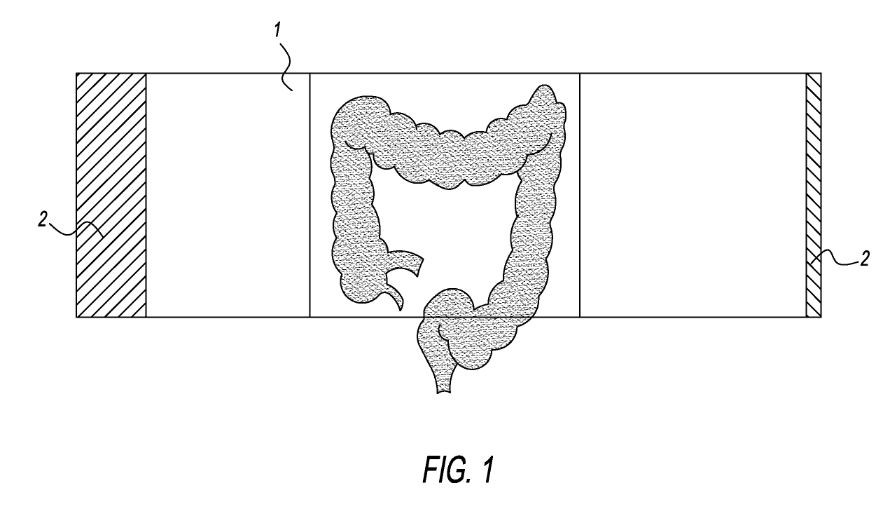

FIG. 1 shows the flexible and breathable elastic abdominal binder 1 which is used to surround the abdomen of a patient undergoing a colonoscopy. The length of the binder 1 can be from 45 inches to 96 inches in order to adapt to the abdominal girth of the patient. The width of the binder 1 can be from 4 inches to 16 inches to adapt to the patient's physique. In one embodiment, the preferred width of the binder 1 is 6 inches. The binder 1 is secured around the abdomen of the patient by any fastening means 2 on each end that couples to each other. In one embodiment, the preferred fastening means 2 is a soft hook and loop fastener Velcro® at one end of the binder and a hard hook and loop fastener such as Velcro® at the other end of the binder. The binder 1 is made out of flexible, breathable elastic materials known in the art.

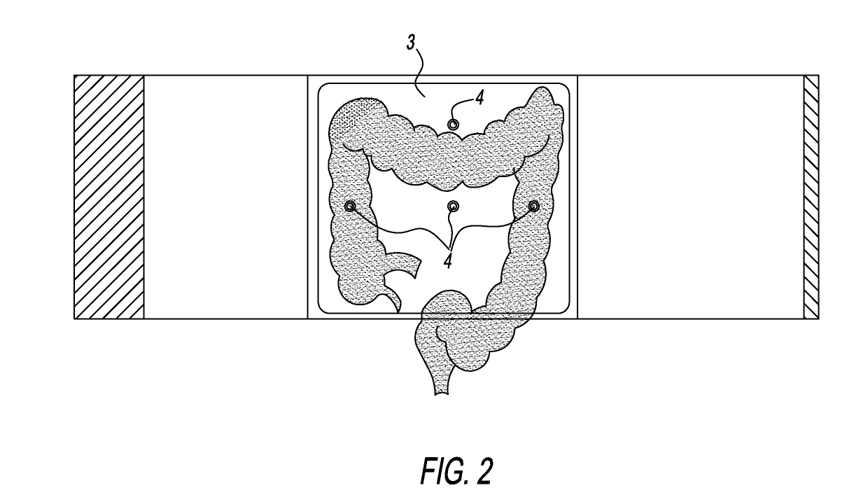

The counter pressure plate 3 as shown in FIG. 2 is secured in any manner to the inside middle of the binder 1. The function of the counter pressure plate 3 is to provide counter pressure during and while one or more inflatable bladders are inflated. Thus the counter pressure plate 3 is a hard, supportive plate made out of metal, cardboard, or preferably plastic. It can measure between 2″×2″ to 8″×8″, preferably 6″×6″. FIG. 2 also shows that four ports 4 for each of the respective four inflatable bladders are placed at the 12, 9, 3 o'clock positions and the center of the plate 3. These ports are the opening in the plate 3/binder 1 to allow connection between the plate 3/binder 1 and the inflatable bladders 6, 7, 8, 9. The locations of these ports ensure that the bladders 6, 7, 8, 9 will be placed over the critical regions of a patient's abdomen.

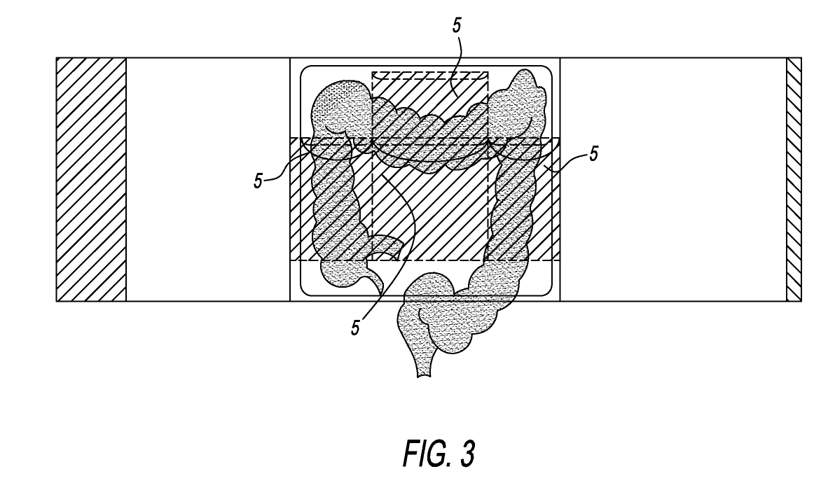

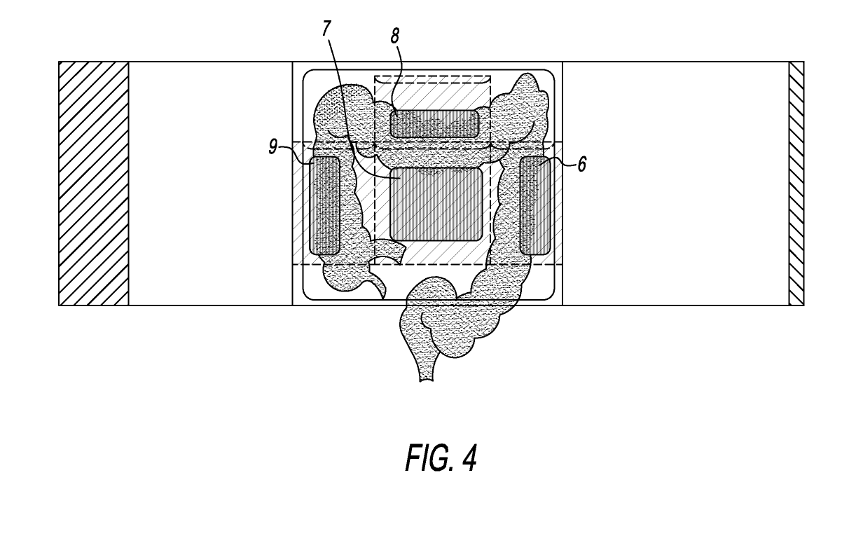

Four bladder sleeve pockets 5 are also placed onto the plate 3 as shown in FIG. 3 in any proper manner to accommodate each of the respective four inflatable bladders 6, 7, 8, 9 as illustrated in FIG. 4. The bladder sleeve pockets can be of any shape, made of any elastic materials and can be removable.

An inflatable bladder 6, 7, 8, 9 can be of any shape, such as round, square, rectangular, oval, oblong, or triangular, can hold air or liquid, and can be made of medical grade PVC, rubber, or plastic, but preferably non-latex rubber.

The inflatable bladders can be between 1″ to 8″ in length. Each of the four inflatable bladders 6, 7, 8, 9 may be of a different shape. The four inflatable bladders 6, 7, 8, 9 are respectively placed into the four bladder sleeve pockets 5.

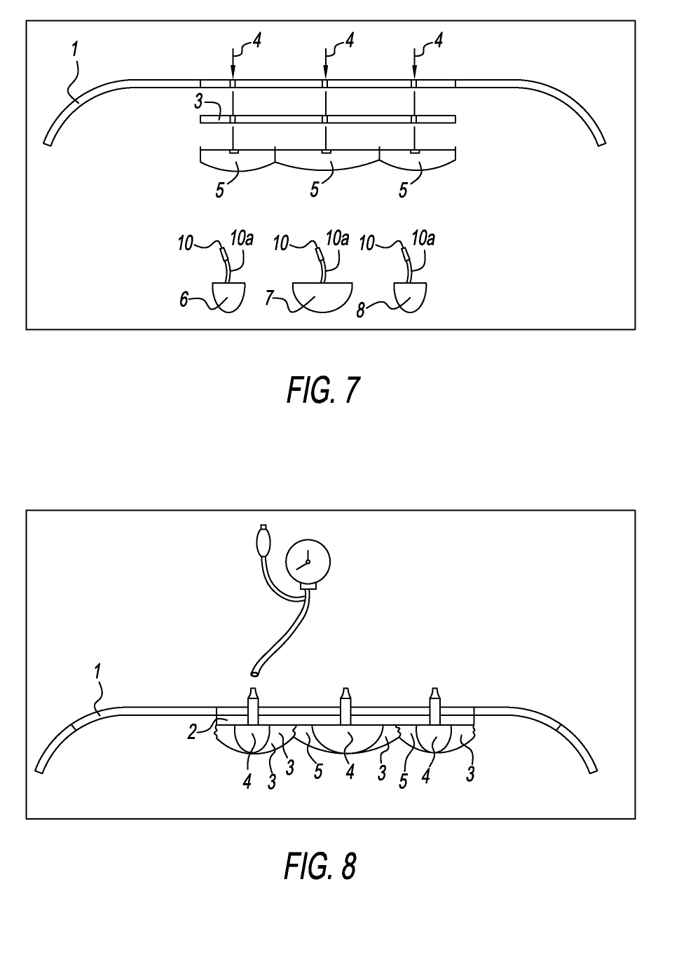

As demonstrated in FIG. 6, each of the inflatable bladders 6, 7, 8, 9 has a tube 10a and a male tube/valve 10 extending through the slot in the sleeve pocket 5 and one of the ports 4 in the plate 3/binder 1, and reaching the female end of an aneroid or hydrostatic manometer 11. The aneroid or hydrostatic manometer 11 with tubing and inflation bulb is attached to the air control/release valve 10 of an inflatable bladder 6, 7, 8, 9 in order to inflate the bladder. The inflation bulb may be either hand held, mechanical, or stationary, but preferably hand held. Air or liquid is inflated to each of the corresponding four inflatable bladders 6, 7, 8 9 while observing the manometer 11 for pressure control. Each of the four inflatable bladders 6, 7, 8, 9 may be inflated to different pressures depending on the patient. The detailed connections between the inflatable bladders 6, 7, 8, 9 and the plate 3/binder 1 and the manometer 11 are further illustrated in FIG. 7 and FIG. 8.

FIG. 7 illustrates a side view of the pneumatic compressor 100 wherein the inflatable bladders 6, 7, 8, 9 are disconnected from the manometer 11. FIG. 7 shows the manometer having a tube with a female end adapted for connecting with the air control release male valves 10 on the inflatable bladders 6, 7, 8, 9. The inflatable bladders 6, 7, 8, 9 are not yet inserted into the sleeve pockets 5. The expandable sleeve pockets 5 are attached to the plate 3 which is then attached to the binder 1.

FIG. 8 is a side view of the pneumatic compressor 100 wherein the inflatable bladders 6, 7, 8, 9 are connected with the manometer 11. The inflatable bladders 6, 7, 8, 9 are inside the sleeve pockets 5. The air control release male valves 10 on the inflatable bladders 6, 7, 8, 9 are inserted through ports 4 in the binder 1/plate 3 to meet the female end of the manometer 11 for air supply.

Returning now to FIG. 4, when the binder 1 is secured to the abdomen, the four un-inflated inflatable bladders 6, 7, 8, 9 will be over four noted regions of the abdomen corresponding to the underlying colon anatomy, such as strategic areas of the colon that commonly loop during a colonoscopy. For example, the four noted regions of the abdomen may correspond to the ascending colon, the mid-umbilical colon, the transverse colon, and the sigmoid bladder.

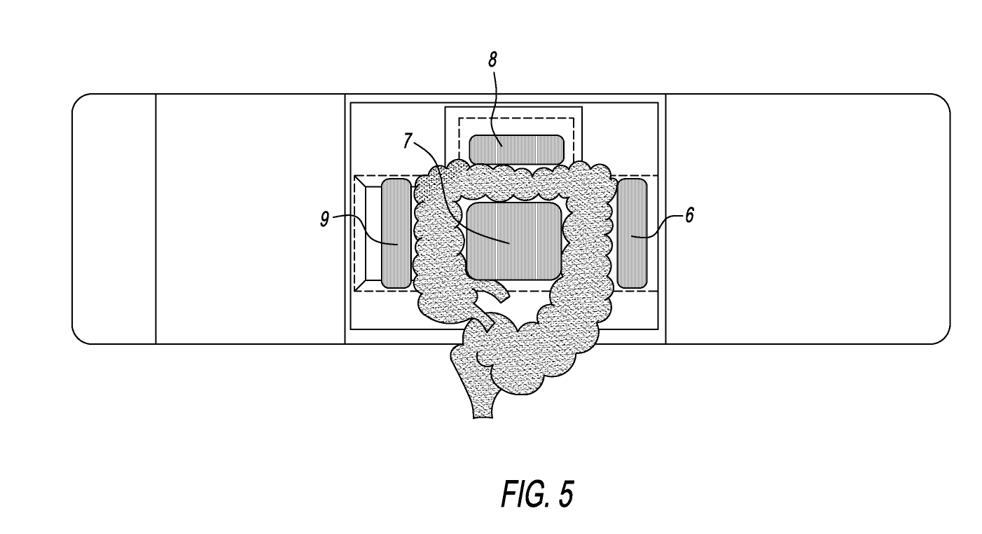

The four inflatable bladders 6, 7, 8, 9 while inflated apply a downward pressure to specific anatomical areas of the large intestine as depicted in FIG. 5, thus preventing the colon from taking the path of least resistance around turns. The large intestine is maneuvered into shape to accept advancing colonoscope and to decrease and stabilize looping throughout procedure.

The binder 1 and counter pressure plate 3 provide a counter pressure during and while the bladder or bladders are inflated. Each bladder 6, 7, 8, 9 can be manually regulated by a manometer 11 by using the air control/release valves 10, and the pressure can be controlled throughout a colon examination at the will of a physician.

It should be understood, of course, that the foregoing relates to exemplary embodiments of the invention and that modifications may be made without departing from the spirit and scope of the invention as set.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. An external pneumatic compression device for applying pressure to a patient's abdomen, comprising:
a) a flexible and breathable elastic abdominal binder having a fastening means on each of two ends for attaching one end to the other;
b) a plurality of inflatable bladders for applying downward pressure to specific anatomical areas of the large intestines of a patient when inflated;
c) a counter pressure plate that is secured to the middle inside of the binder, providing a counter pressure during and while the bladder or bladders are inflated; and
d) a means for supplying air and regulating the pressure in the bladders.

2. The compression device of claim 1 further comprising a plurality of air control/release valves that are attached to the plurality of inflatable bladders.

3. The compression device of claim 1 further comprising a plurality of ports are placed at the 12, 9, 3 o'clock positions and the center of the counter pressure plate.

4. The compression device of claim 1 further comprising a plurality of bladder sleeve pockets to accommodate a plurality of inflatable bladders.

5. The compression device of claim 1 wherein the fastening means is a soft hook and loop fastener Velcro® at one end of the binder and a hard hook and loop fastener such as Velcro® at the other end of the binder.

6. The compression device of claim 1, wherein the abdominal binder is about 45 to 96 inches in length, and about 4 to 16 inches in width, preferably about 6 inches in width.

7. The compression device of claim 1, wherein the counter pressure plate is made of metal, cardboard, or preferably plastic.

8. The compression device of claim 1, wherein the counter pressure plate can measure about 2″×2″ to 8″×8″, preferably about 6″×6″.

9. The compression device of claim 1, wherein the inflatable bladder is made of medical grade PVC, rubber, plastic, or preferably non-latex rubber.

10. The compression device of claim 1, wherein the inflatable bladder is about 1″ to 8″ in length.

11. The compression device of claim 1, wherein the plurality of inflatable bladders has different shapes, such as round, square, rectangular, oval, oblong, or triangular.

12. The compression device of claim 1, wherein the means for supplying air and regulating pressure is a manometer; the manometer has tubing and a bulb to supply air to the plurality of inflatable bladders.

13. An external pneumatic compression device for applying pressure to a patient's abdomen, comprising:
a) a flexible and breathable elastic abdominal binder having a fastening means on each of two end for attaching one end to the other;
b) a plurality of inflatable bladders for applying downward pressure to specific anatomical areas of the large intestines of a patient when inflated;
c) a plurality of air control/release valves that is attached to the plurality of inflatable bladders;
d) a counter pressure plate that is secured to the middle inside of the binder, providing a counter pressure during and while the bladder or bladders are inflated;
e) a plurality of ports are placed at the 12, 9, 3 o'clock positions and the center of the counter pressure plate;
f) a monometer with tubing and bulbs to attach and control the inflation of the plurality of inflatable bladders; and
g) a plurality of bladder sleeve pockets to accommodate a plurality of inflatable bladders.

14. The compression device of claim 13 wherein the fastening means is a soft hook and loop fastener Velcro® at one end of the binder and a hard hook and loop fastener such as Velcro® at the other end of the binder.

15. The compression device of claim 13, wherein the abdominal binder is about 45 to 96 inches in length, and about 4 to 16 inches in width, preferably about 6 inches in width.

16. The compression device of claim 13, wherein the counter pressure plate is made of metal, cardboard, or preferably plastic.

17. The compression device of claim 13, wherein the counter pressure plate can measure about 2″×2″ to 8″×8″, preferably about 6″×6″.

18. The compression device of claim 13, wherein the inflatable bladder is made of medical grade PVC, rubber, plastic, or preferably non-latex rubber.

19. The compression device of claim 13, wherein the inflatable bladder is about 1″ to 8″ in length.

20. The compression device of claim 13, wherein the plurality of inflatable bladders has different shapes, such as round, square, rectangular, oval, oblong, or triangular.
